(19) United States
(12) Patent Application Publication
(10) Pub. No.: US 2004/0033516 A1
(43) Pub. Date: Feb. 19, 2004

(54) Method for analysing a patient's predisposition to insulin-dependent diabetes, device and set of primers

(76) Inventor: Bruno Mougin, Lyon (FR)

Correspondence Address: James C Lydon, Suite 100, 100 Daingerfield Road, Alexandria, VA, 22314, US

(21) Appl. No.: 10/416,928

(22) PCT Filed: Nov. 16, 2001
(86) PCT No.: PCT/FR01/03599

(30) Foreign Application Priority Data
Nov. 17, 2000 (FR) 00/14896

(51) Int. Cl. C12Q 1/68

(52) U.S. Cl. 435/6

(57) ABSTRACT

This invention concerns a method for testing a subject's predisposition to insulin-dependent diabetes. It also concerns a device suitable for implementation of the method, and a set of primers for amplification for such a method

The method consists in taking a liquid sample containing at least one type of amplicon generated by the amplification of at least one polymorphic region relevant to the disease concerned, and adding to it probes selected in the following way

at least one probe which is specific for the subject's susceptibility to the disease,

at least one probe which is specific for said subject's protection against said disease, and

at least one probe which is specific for said subject's neutral status vis-à-vis predisposition to said disease,

and consisting of visualizing any hybrids formed.

The invention is particularly applicable in the field of diagnosis.

### DESCRIPTION

This invention concerns a method for testing a subject's predisposition to at least one disease referred to as insulin-dependent diabetes.

Type I diabetes (or insulin-dependent diabetes) is a disease which is characterized by destruction of the insulin-secreting β cells of the Islets of Langerhans in the pancreas as the result of an autoimmune process which develops in genetically predisposed individuals. Above and beyond environmental factors, there is a genetic component in predisposition to the disease

Every single individual has his/her own genetic heritage inherited from his/her forebears. This particular genetic background can sometimes actively participate in either the appearance or development of certain health problems: infections by pathogenic agents (e.g. the Human Immunodeficiency Virus) and autoimmune diseases (e.g. rheumatic conditions). Genes in the Major Histocompatibility Complex (MHC), especially those encoding the Human Leukocyte Antigens (HLA) play a particularly important role in the development of autoimmune diseases:

those that do not target any organ in particular e.g. joint diseases like rheumatoid arthritis (Lawrence, 1970; Stastny, 1978; Khan, 1979; Stastny, 1983; Gregersen, 1986; Gregersen, 1987; Stastny, 1988; Todd, 1988; Wordsworth, 1989; Nepom, 1989; Hiraiwa, 1990; Nepom, 1991) and ankylosing spondylitis (Brewerton, 1973; Schlosstein, 1973; Benjamin, 1990), or

organ-specific diseases, more precisely those that attack endocrine glands such as the pancreas, which target is associated with insulin-dependent diabetes (Komulainen, 1999; Kulmala, 2000).

A large fraction of the genetic component of predisposition to insulin-dependent diabetes has been attributed to one main marker located in the HLA locus (IDDM1), and more precisely in the genes encoding HLA-DQB1. Published results now make it possible to define the most common of the forty-five (45) HLA-DQB1 alleles inventoried in the international classification system as conferring either susceptibility (S alleles) or protection (P alleles), or as being neutral neutral (N alleles) (www.anthonynolan.com/HIG/seq/nuc; octobre 2000). The results have mainly been confirmed in Caucasian populations. At this time, the molecular hypothesis is based on a polymorphism observed at amino acid 57 in the β chain of the HLA-DQ protein (Todd et al., 1988). Table 1 below summarizes current knowledge about status vis-à-vis insulin-dependent diabetes as it depends on the nature of the amino acid at position 57.

TABLE 1
Status vis-à-vis insulin-dependent diabetes as it
depends on the nature of the amino acid at position 57

| Status vis-à-vis insulin- | | Amino acid at |
|---|---|---|
| dependent diabetes | HLA-DQB1allele* | position 57 |
| S | 02 | Alanine (A) |
| | 0302 | |
| P | 0301 | Aspartic acid (D) |
| | 0602 | |
| | 0603 | |
| N | Others | Valine (V) or |
| | | Serine (S) |

Each person's genotype consists of a combination of two alleles. Thus, each possible combination is associated with a differential predisposition to Type 1 diabetes: SS, PP, NN, SP, SN or NP. This molecular hypothesis is consistent with the observation that the disease is more severe in people carrying two S alleles than in people carrying only one, and more severe in those with one S allele than in those with none. This is commonly referred to as a dose effect.

A recent article by Cinek et al. entitled <<Screening for the IDDM high-risk genotype. A rapid microtitre plate method using serum as source of DNA>>, Tissue Antigens, 2000, 56, 4, 344-349 drew attention to the need to develop a test to analyze genetic predisposition to insulin-dependent diabetes. This team chose an approach based on serum which is comprehensive for the alleles in question (certain HLA-DQB1 alleles and also some HLA-DQA1 and some HLA-DRB1*04 alleles).

A serum-based test is far less practical than a protocol based on a spot of dry blood. Moreover, their method is complicated, in that:

the test necessitates two different steps, the first to analyze the HLA-DQB1 and HLA-DQA alleles, and the second to analyze the HLA-DRB1*04 alleles,

it uses a complicated technique to immobilize the capture probes, with a hybridization temperature of 63° C., washes first at 63° C. and then at 18-25° C., and two-step development with streptavidin-peroxidase (SPOD).

Moreover, this method cannot in any way detect the dose effect which nevertheless has a major impact on a subject's predisposition to insulin-dependent diabetes.

Finally, these tests can only be performed in laboratories with expertise in the analysis of HLA genes (i.e. blood banks and certain specialist hospitals). In consequence, the test necessarily involves transporting a sample and requires relatively sophisticated equipment. Thus, the negative consequences are multiple, including:

the risk that a sample will be lost,

the turnaround time of the identification test will be long,

said identification test will be relatively expensive, and

the person ordering the test has no control over the service provider.

The claimed test method affords a test which is simpler in practical terms, faster (complete in less than two hours after preparation of the amplicons) and easy to perform.

To this effect, this invention concerns a method for testing for a subject's genetic predisposition to an autoimmune disease, consisting of taking a liquid sample containing at least one type of amplicon generated by the amplification of at least one polymorphic region relevant to the disease concerned, and adding to it probes selected in the following way:

at least one probe which is specific for the subject's susceptibility to the disease,

at least one probe which is specific for said subject's protection against said disease,

at least one probe which is specific for said subject's neutral status vis-à-vis said disease,

and consisting of visualizing any hybrids formed.

Preferably, this method is used to test for a subject's predisposition to insulin-dependent diabetes.

According to a modified embodiment, the probes used to detect subjects' status vis-à-vis insulin-dependent diabetes are defined as follows:

at least one probe which is specific for the susceptibility alleles HLA-DQB1*0201, HLA-DQB1*0202 and HLA-DQB1*0302,

at least one probe which is specific for the protective alleles HLA-DQB1*0301, HLA-DQB1*0602 and HLA-DQB1*0603,

at least one probe which is specific for other alleles which are neutral vis-à-vis a subject's predisposition to insulin-dependent diabetes.

According to a modified embodiment, the probes used to detect subjects' status vis-à-vis insulin-dependent diabetes are defined as follows:

at least one probe which is specific for the susceptibility alleles HLA-DQB1*0201, HLA-DQB1*0202, HLA-DQB1*0203, HLA-DQB1*0302, HLA-DQB1*0304, HLA-DQB1*0305, HLA-DQB1*0307 and HLA-DQB1*0308,

at least one probe which is specific for the protective alleles HLA-DQB1*03011, HLA-DQB1*03012, HLA-DQB1*03032, HLA-DQB1*03033, HLA-DQB1*0304, HLA-DQB1*0306, HLA-DQB1*0308, HLA-DQB1*0309, HLA-DQB1*0310, HLA-DQB1*0602, HLA-DQB1*0603, HLA-DQB1*0608, HLA-DQB1*0610, HLA-DQB1*06111, HLA-DQB1*06112, HLA-DQB1*0612, HLA-DQB1*0613, HLA-DQB1*0614 and HLA-DQB1*0616, and at least one probe which is specific for the neutral alleles HLA-DQB1*0306, HLA-DQB1*0401, HLA-DQB1*0402, HLA-DQB1*05011, HLA-DQB1*05012, HLA-DQB1*0502, HLA-DQB1*05031, HLA-DQB1*05032, HLA-DQB1*06011, HLA-DQB1*06012, HLA-DQB1*06013, HLA-DQB1*06051, HLA-DQB1*06052, HLA-DQB1*0606, HLA-DQB1*0609, HLA-DQB1*06112 and HLA-DQB1*0612.

More precisely and in all the above-mentioned modifications, the probes used to detect susceptibility to insulin-dependent diabetes are defined as follows:

a probe which is specific for the alleles HLA-DQB1*0201, HLA-DQB1*0202 and HLA-DQB1*0203, and

a probe which is specific for the alleles HLA-DQB1*0302, HLA-DQB1*0304, HLA-DQB1*0305, HLA-DQB1*0307 and HLA-DQB1*0308.

According to this latter case, the probes used to detect susceptibility to insulin-dependent diabetes consist of at least ten (10) nucleotides linked to form the following sequences:

| | |
|---|---|
| (TCTTgTgAgCAgAAgC), and | SEQ ID NO 5 |
| (CCgCCTgCCgCCgA). | SEQ ID NO 6 |

More precisely and in all the above-mentioned modifications, the probes used to detect protection against insulin-dependent diabetes are defined as follows:

a probe which is specific for the alleles HLA-DQB1*03011, HLA-DQB1*03012, HLA-DQB1*0304 and HLA-DQB1*0309, and

a probe which is specific for the alleles HLA-DQB1*03011, HLA-DQB1*03012, HLA-DQB1*03032, HLA-DQB1*03033, HLA-DQB1*0306, HLA-DQB1*0309 and DQB1*0310, and

a probe which is specific for the alleles HLA-DQB1*0308, HLA-DQB1*0602, HLA-DQB1*0603, HLA-DQB1*0608, HLA-DQB1*0610, HLA-DQB1*06111, HLA-DQB1*06112, HLA-DQB1*0612, HLA-DQB1*0613, HLA-DQB1*0614 and HLA-DQB1*0616.

According to this latter case, the probes used to detect protection against insulin-dependent diabetes consist of at least ten (10) nucleotides linked to form the following sequences:

| | |
|---|---|
| (AggggACCCgggCggA), | SEQ ID NO 7 |
| (gACgTggAggTgTACC), and | SEQ ID NO 8 |
| (gCCgCCTgACgCCg). | SEQ ID NO 9 |

More precisely and in all the above-mentioned modifications, the probes used to detect neutrality vis-à-vis a subject's predisposition to insulin-dependent diabetes are defined as follows:

a probe which is specific for the alleles HLA-DQB1*0306, HLA-DQB1*0401 and HLA-DQB1*0402, and

a probe which is specific for the alleles HLA-DQB1*05011, HLA-DQB1*05012, HLA-DQB1*0502, HLA-DQB1*05031 and HLA-DQB1*05032,

a probe which is specific for the alleles HLA-DQB1*06011, HLA-DQB1*06012 and HLA-DQB1*06013, and

a probe which is specific for the alleles HLA-DQB1*06051, HLA-DQB1*06052, HLA-DQB1*0606, HLA-DQB1*0609, HLA-DQB1*06112 and HLA-DQB1*0612.

According to this latter case, the probes used to detect neutrality vis-à-vis a subject's predisposition to insulin-dependent diabetes consist of at least ten (10) nucleotides linked to form the following sequences:

| | |
|---|---|
| (ggggCCCgggCgTC), | SEQ ID NO 10 |
| (AggAggACgTgCgC), | SEQ ID NO 11 |
| (TCTTgTAACCAgATAC), and | SEQ ID NO 12 |
| (ggTggACACCgTATgCAg). | SEQ ID NO 13 |

In all cases, at least one positive control probe capable of hybridizing with all HLA-DQB1 genes is used to detect all HLA-DQB1 alleles.

Moreover, up to 38.89%, preferably no more than 20%, of the bases in a given probe are substituted by at least one base analog such as inosine. The above figures can be deduced from a prior patent application submitted by the Applicant under Number PCT FR00/01385 under priority of Jun. 20, 1999 and Dec. 6, 2000. The term analog base is defined further on in this Description.

Prior to testing for a subject's genetic predisposition to an autoimmune disease as described above, at least one round of amplification of the relevant polymorphic region or regions in the HLA-DQB1 locus is carried out.

Preferably, the primers used for the amplification reaction are biotinylated so that the resultant amplicons will be likewise biotinylated.

Prior to amplification, the biological sample (preferably in the form of a dry spot of blood) is processed to extract its nucleic acids.

Nucleic acids are extracted into a reaction mixture which already contains the deoxynucleotide triphosphates (dNTPs) to be used in the amplification reaction, and this prior to incubation.

In this latter case, after incubation, deoxynucleotide triphosphates (dNTPs)—also to be used for the subsequent amplification step—are added into the reaction mixture.

This invention also concerns a device for implementing a method as described above, in which according to a first embodiment, each type of specific probe is immobilized in a separate compartment (e.g. the well of a microtiter plate), apart from the others.

According to a second embodiment, each type of probe used to detect susceptibility to or protection against insulin-dependent diabetes is immobilized in a separate compartment (e.g. the well of a microtiter plate), apart from the other probes used to detect susceptibility or protection, and all or a fraction of the various types of probe used to detect neutrality vis-à-vis a subject's predisposition to insulin-dependent diabetes is immobilized in at least one well of a microtiter plate.

According to a third embodiment, at least two types of different, specific probe are immobilized in a single compartment (e.g. the same well of a microtiter plate) without any interaction occurring between them.

According to a fourth embodiment, all the different types of probe used to detect susceptibility to, protection against, and neutrality vis-à-vis insulin-dependent diabetes are immobilized in a single compartment (e.g. the same well of a microtiter plate).

In the case in which one compartment (e.g. one well of a microtiter plate) contains only one type of probe used to detect either susceptibility or protection, any hybrids formed in the device are visualized by an unlocalized, enzyme-catalyzed calorimetric reaction. For example, this could mean visualizing hybrids formed between each type of probe and the amplicons using peroxidase.

In the case in which one compartment (e.g. one well of a microtiter plate) contains at least two types of probe used to detect either susceptibility or protection, the method used to visualize the hybrids formed with a device would involve a localized reaction. For example, this could mean visualizing hybrids formed between each type of probe and the amplicons using a fluorescent or radioactive label.

The invention finally concerns a set of primers for the amplification of a sequence corresponding to the HLADQB1 gene, designed for use in a method to test for a subject's genetic predisposition to insulin-dependent diabetes, which involves using a SEQ ID NO 1 primer in conjunction with a SEQ ID NO 2 primer.

Preferably, the primers are biotinylated at the 5′ end.

Moreover, the capture probes which can hybridize with sequences corresponding to the HLA-DQB1 gene designed for the testing of a subject's genetic predisposition to insulin-dependent diabetes, are immobilized either at the bottom of the well of a microtiter plate or on a bead through an amine or biotin bridge located at the 5′ end of said probes.

The accompanying examples are given for the purposes of illustration and are not to be taken as limiting in any way. They are designed to make the invention easier to understand.

This invention concerns a method for detecting genetic diseases which is fast and cheap. This new technology can be exploited for all genetic diseases, and particularly for rheumatoid arthritis, ankylosing spondylitis, insulin-dependent diabetes and other autoimmune diseases such as those which attack connective tissue (lupus, scleroderma, etc.) which are also encountered in the practice of rheumatology.

It is a special method for testing an individual's genetic predisposition to certain diseases, based on a technique of molecular biology using which at least one gene can be tested for simultaneously. This method can be advantageously used to test an individual's genetic predisposition to a disease or set of related diseases associated with one or more genes. This method can be used to test an individual's genetic predisposition to certain organ-specific, autoimmune diseases, e.g. insulin-dependent diabetes in which the target organ is the pancreas.

The advantage of this method is that it gives in one step, with a single but pluripotential test, a complete set of important, clinically relevant information (useful for diagnosis, prognosis and therapeutic guidance). The method involves a number of distinct steps:

1) extraction of nucleic acid from the individual's biological sample,

2) amplification of the sequences of interest in which a disease-related polymorphism has been reported, and

1) simultaneous analysis of the amplicons using a series of hybridization reactions based on a set of molecular probes designed for the precise testing of specific alleles or groups of alleles.

I—Definitions

Compartment means any flat or concave (i.e. reservoir-forming) solid substrate, which is capable of holding either:

in the first case, an aliquot of liquid in such a way that said liquid does not interact with other aliquots of the same or other liquids present in any neighboring compartment,

in the second case, several aliquots of the same liquid or different liquids, of identical or different volumes, in such a way that none of these aliquots interact with other aliquots of the same or other liquids present in any neighboring compartment

A device which dispenses these aliquots of liquid, the volume of which may be between hundreds of microliters (for the first case) and 0.5 nanoliters (in the second case), as well as the method implemented in such a device and the substrates embodied for the test in the device have all already been described in a Patent Application submitted on Nov. 15, 2000 by the Applicant under N^{o} FR00/14691.

In the second case, the various volumes of liquid dispensed into a single compartment are very small, generally less than one microliter, and they form spots on the surface of said compartment. The volume of solution in each drop is between 0.5 nanoliters and 1 microliter, preferably between 1 nanoliter and 200 nanoliters

The term solid substrate as used in the preceding definition includes all materials on which an analyte—in this case a nucleic acid—an be immobilized. Natural and synthetic materials (chemically modified or not) can be used to make a solid substrate, notably polymers such as polyvinyl chloride, polyethylene, polystyrene, polyacrylate and polyamide, or copolymers made from aromatic vinyl monomers, alkylesters of α-unsaturated or β-unsaturated acids, esters of unsaturated carboxylic acids, vinylidene chloride, dienes or compounds containing nitrile groups (acrylonitrile); polymers of vinyl chloride and propylene, the polymer of vinyl chloride and vinyl acetate, copolymers based on styrenes or substituted styrene derivatives; synthetic fibers such as nylon, nitrocellulose; inorganic materials such as silica, glass, ceramics and quartz; latex; magnetic beads, metal derivatives.

The solid substrate according to the invention may be—without any limitation—in the form of a microtiter plate, a sheet, a tube, a well, beads, or a flat substrate such as a wafer made of silica or silicon. Preferably, at least one part of the substrate is either flat (e.g. a silicon wafer) or forms the bottom of the well of a microtiter plate. Preferably, this compartment consists of the well of a microtiter plate.

The solid substrate may be hydrophilic and/or hydrophobic, depending on the application envisaged and the nature of the analyte-containing solution. For example, liquids could be deposited onto a hydrophilic patch surrounded by a hydrophobic area thereby providing control over the diameter of the spots.

Visualizing hybrids means that use is made of a polynucleotide labelled with a marker reagent. The term marker reagent refers to a tracer which generates a signal which can be detected, either directly or indirectly. A non-limiting list of such markers follows below:

enzymes, such as horseradish peroxidase, alkaline phosphatase, β-galactosidase and glucose-6-phosphate dehydrogenase which generate a detectable signal when an appropriate substrate is added, with detection by means of colorimetry, fluorescence or luminescence, or

chromophores such as fluorescent, luminescent or colored compounds, or

electron-dense species which can be visualized by electron microscopy or detected by virtue of some electrical parameter, e.g. conductance, current, potential difference or impedance, or

species which can be detected using techniques exploiting either optical properties (such as diffraction, surface plasmon resonance or contact angle variation), or physical properties (such as atomic force spectroscopy or the tunneling effect), or

radioactive species such as ³²P, ³⁵S or ¹²⁵I.

Preferably, the marker is a fluorescent compound which does not cause significant steric hindrance, such as fluorescein, dansyl, IR chromophores (Li-COR Inc, Lincoln Nebr., USA), Cy5 and Cy3 (Randolph J. B. et al., Nucleic Acids Res., 25(14), p2923-2929, 1997) and derivatives thereof. Compounds which do not cause significant steric hindrance should be taken as having a molecular weight of below 1000 g/mol.

The method involves detection of the target nucleic acid in a sample by placing said target nucleic acid—after any necessary pre-treatment—with, among other things, the derivatized nucleotide in order to synthesize a derivatized polynucleotide, then labeling said derivatized polynucleotide with the marker reagent in order to make it possible to detect said labeled polynucleotide.

Pre-treatment refers to the various processes to which a sample is subject in order to render the target nucleic acid accessible, such as for example lysis, liquefaction and concentration.

“At least one probe which is specific for other alleles which are neutral vis-à-vis a subject's predisposition to insulin-dependent diabetes” refers to at least one probe which is specific for all or a fraction of HLA-DQB1 alleles which have not been defined as conferring either susceptibility to or protection against insulin-dependent diabetes. In general, the probes are selected on the basis of the known prevalence of the alleles; thus, the more common a neutral allele, the more important it is to include the corresponding specific probe.

Another component of the test may involve at least one probe—either on its own or in conjunction with at least one of the above-mentioned probes—which is specific for all or a fraction of the alleles comprising the HLA-DR3 locus and/or the HLA-DR4 locus. Thus, a large number of articles concerning these haplotypes may be relevant:

Park Y. S., She J. X., Noble J. A., Erlich H. A. et Einsenbarth G. S., Tissue Antigens 2001: 57: 185-191: <<Transracial evidence for the influence of the homologous HLA DR-DQ haplotype on transmission of HLA DR4 haplotypes to diabetic children>>,

Kockum I., Sanjeevi C. B., Eastman S., Landin-Olsson M., Dahlquist G., Lernmark A. et al., European Journal of Immunogenetics 26, 361-372: <<Complex interaction between HLA DR and DQ in conferring risk for childhood type 1 diabetes >>,

Undlien D. E., Kockum I., Ronningen K. S., Lowe R., Sanjeevi C. B., Graham J., Lie B. A., Akselsen H. E., Lernmark A. and Thorsby E., Tissue Antigens 1999: 54, 543-551: <<HLA associations in type A diabetes among patients not carrying high-risk DR3-DQ2 or DR4-DQ8 haplotypes >>,

Donner H., Seidl C., Van der Auwera B., Braun J., Siegmund T., Herwig J., Weets I., The Belgian Diabetes Registry, Usadel K. H. et Badenhoop K., Tissue Antigens 2000: 55: 271-274: <<HLA-DRB1*04 and susceptibility to type 1 diabetes mellitus in a German/Belgian family and German case-control study >>,

Redondo M. J., Kawasaki E., Mulgrew C. L., Noble J. A., Erlich H. A., Freed B. M., Lie B. A., Thorsby E., Einsenbarth G. S., Undlien D. E., Kockum I. et Ronningen K. S., The Journal of Clinical Endocrinology & Metabolism, 2000: Vol.55, No 10: 3793-3797: <<DR- and DQ-Associated Protection from Type 1A Diabetes: Comparison of DRB1*1401 and DQA1*0102-DQB1*0602 >>.

Amplification means that the derivatized nucleotides are synthesized in an enzyme-mediated amplification reaction in which the target nucleic acid acts as the template. Articles by Lewis (1992. Genetic Engineering News, 12, p. 1-9) and Abramson and Myers (1993. Curr. Opin. Biotechnol., 4, p. 41-47) both give examples of target amplification. The enzyme-mediated amplification method used can be selected from among the following: NASBA (Nucleic Acid Sequence Based Amplification), TMA (Transcription Mediated Amplification) RT-PCR (Reverse Transcriptase-Polymerase Chain Reaction), PCR (Polymerase Chain Reaction), SDA (Strand Displacement Amplification) and LCR (Ligase Chain Reaction).

Analog base means a modified nucleotide which is usually incorporated into a polynucleotide. A polynucleotide consists of a sequence of at least two deoxyribonucleotides or ribonucleotides and may include at least one nucleotide containing a modified base such as inosine, methyl-5-deoxycytidine, dimethylamino-5deoxyuridine, deoxyuridine, diamino-2,6-purine, bromo-5-deoxyuridine, nebularine or any other modified base which does not block hybridization. The polynucleotide may also be modified at:

the internucleotide linkage, e.g. the phosphorothioates, the H-phosphonates and the alkyl-phosphonates, or

the backbone, e.g. the α-oligonucleotides ( FR-A-2.607.507) ou les PNA (M. Egholm et al., J. Am. Chem. Soc., 114, p1895-1897, 1992) or the 2′ O-alkyl riboses.

Any of these modifications may be combined. The polynucleotide may be an oligonucleotide, a naturally occurring nucleic acid, a fragment thereof, e.g. of DNA, of a ribosomal RNA, of a messenger RNA, of a transfer RNA, or a nucleic acid molecule generated in an enzyme-mediated amplification reaction.

II—Sample Preparation

1^{o}) Extraction of Deoxyribonucleic Acid (DNA)

A—From a Sample of Whole Blood

Any of the various classic protocols for extracting DNA from whole blood drawn into an anticoagulant such as EDTA (ethylenediaminetetraacetic acid), citrate or heparin can be used. The protocol may involve a phenol extraction step, or successive extraction of first red blood cells and then leukocytes (Kimura et al., 1992).

In practice and according to one embodiment, extraction is carried out using an entirely classic method. In fact, any method can be used to extract the DNA as long as the resultant material can be subsequently amplified in an amplification process such as the Polymerase Chain Reaction (PCR). These cell lysis methods involving extraction followed by purification of nucleic acid are usually those recommended for genetic tests or rapid tests using commercially available products, e.g. the QIAmp Blood Kit (Registered Trademark) sold by QIAGEN S.A.

B—From Spots of Dry Blood

The literature descries various methods for extracting DNA from dried blood samples.

The first is the method of Jinks et al., Hum. Genet. 1989, 81: 363-366. in which blood is spotted onto Schleicher & Schuell paper (no. 903). The spots are allowed to dry for several hours at a temperature of between 18 and 25° C. Four wafers are cut out, each with a diameter of 3 mm, and are placed in a 1.5 ml tube. Fixation is carried out with 30 μl of methanol which is then evaporated off. A 60 μl aliquot of water is added to the tube and boiled for 15 minutes. Then the tube is centrifuged (10,000 g) for 15 minutes. Finally, the supernatant is drawn off for the next stage, i.e. PCR amplification.

The second method is that of Hezard et al., Thrombosis Research, 1997, 88, 1, 59-66. Blood drawn into EDTA, citrate or heparin is spotted onto a Guthrie filter paper. A 1 mm wafer of the filter paper is placed in 50 μl of the reaction mixture for later amplification. It is incubated for 15 minutes at 94° C. Finally, the Taq polymerase is added and PCR amplification is performed.

In the year 2000, the Molecular Innovations Inc. company put a DNA extraction kit on the market; this represents the third method. The entire process is completed in a single amplification tube (Xtra Amp [Registered Trademark] Extraction System, Molecular Innovations Inc., Ref. 660). We have tested this kit with respect to extracting DNA from spots of dry blood The blood is drawn into EDTA or ACD and spotted onto Schleicher & Schuell paper, ref. 322187. A 3 mm wafer is placed in a 1.5 ml microfuge tube containing 75 μl of Lysis Buffer. This is then incubated for 10 minutes at a temperature of between 18 and 25° C. Then the tube is centrifuged (12,000 g) for 5 minutes. The supernatant is then drawn off and transferred into an Xtra Amp (Registered Trademark) tube. After several cycles of aspiration and expulsion of the liquid back into the tube, the supernatant is discarded. It is then washed three times with 200 μl aliquots of Wash Buffer. Then 45 μl of the PCR amplification reaction mixture are added together with 5 μl of Amp Enhance Buffer. Then amplification is performed with three more cycles than the usual program.

Using all three protocols described above, we obtained adequate HLA-DQB1 amplification for the ELOSA (Enzyme Linked OligoSorbent Assay) as developed in this invention, in about 50% of cases. The ELOSA method is thoroughly described in the Applicant's Patent EP-B-0.486.661 and in the article by F. Mallet et al., Journal of Clinical Microbiology, June 1993, p.1444-1449.

However, a more robust extraction protocol for spots of blood needs to be developed for routine testing applications. In consequence, the Applicant has developed a fourth method which comprises the steps of:

drawing blood into EDTA, spotting it onto Schleicher & Schuell paper (no. 903, ref. 322187),

cutting out 3 mm wafers of the spot using a Schleicher & Schuell punch,

decontaminating the punch with 0.25 N HCl for 5 minutes (which depurinates the DNA), after the cutting of each sample of blood,

placing the wafer in a 0.5 ml PCR tube,

adding 50 μl of a reaction mixture which consists of 5 μI of a ten-fold concentrated (10×) amplification buffer plus 0.5 μl of the NTP mixture (200 mM) plus H₂O to bring the total volume to 50 μl,

incubating 15 minutes at 100° C. (using a thermocycler is recommended),

removing the wafer,

a brief centrifugation (1,000 g) in a standard bench-top microcentrifuge, and

drawing off 25 μl of the supernatant for the amplification reaction.

Surprisingly, the reaction mixture used to resuspend the blood on each wafer already contains the deoxyribonucleotide triphosphates (dNTPs), namely DATP, dCTP, dGTP and dTTP. These correspond to biological species which those skilled in the art tend to try to avoid exposing to high temperatures. However, in the protocol developed by the Applicant, the temperature of said reaction mixture is kept at 100° C. for a period of 15 minutes. The results of the amplification of nucleic acid extracted by means of this method are far better than those obtained with any of the first three methods, as described above.

2^{o}) Preparation of the HLA-DQB1 Amplicons

In all cases, the primers used are those described in the literature (XIth HLA Workshop Primers; Kimura, 1992). Table 2 below gives the sequence of the two primers used to amplify the locus corresponding to the HLA-DQB1 gene. These two primers flank said gene.

TABLE 2

Primers for amplification of the HLA-DQB1 gene

| | | |
|---|---|---|
| Sequence n° | Sequence (5′ > 3′) | Scientific name |

| | | |
|---|---|---|
| SEQ ID NO 1 | CATgTgCTACTTCACCAACgg | DQBAMP-A |
| SEQ ID NO 2 | CTggTAgTTgTgTCTgCACAC | DQBAMP-B |

A—From a Sample of Whole Blood

The reaction mixture contains the following components:

| | |
|---|---|
| 10X buffer (Perkin Elmer, ref. N 808-0171) | 5 μl, |
| dNTPs (200 mM) (Pharmacia, ref. 27-2094) | 0.5 μl (final concentration: 0.2 mM), |
| primer mixture (containing 30 μM of each) | 0.5 μl (final concentration: 0.3 μM), |
| Taq Polymerase (AmpliTaq, Perkin Elmer, ref. N 808-0171, 5 U/μl) | 0.3 μl (1.5 U), |
| DNA (about 100 ng/μl) | 5 μl (about 500 ng), and |
| H₂O | q.s. 50 μl. |

The amplification program is as follows:

2 minutes at 95° C., then

35 successive cycles, each cycle comprising the following steps:

30 seconds at 95° C.,

30 seconds at 55° C., and

30 seconds at 72° C.,

7 minutes at 72° C.

Then, if the product of the reaction is to be stored, the tube containing the amplicons which were just synthesized is kept at a temperature of 9° C.

B—From Spots of Dry Blood

The reaction mixture contains the following components:

| | |
|---|---|
| 10X buffer (Perkin Elmer, ref. 27-2094) | 5 μl, |
| dNTPs (200 mM) (Pharmacia, ref. N 808-0171) | 0.5 μl, |
| primer mixture (containing 30 μM of each) | 0.5 μl (final |
| | concentration: |
| | 0.3 μM), |
| Taq Polymerase (AmpliTaq, Perkin Elmer, | 0.3 μl (1.5 U), |
| ref. N 808-0171, 5U/μl). | |
| DNA 25 μl, and | |
| H₂O | q.s. 50 μl. |

The amplification program is as follows:

2 minutes at 95° C., then

35 successive cycles, each cycle comprising the following steps:

30 seconds at 95° C.,

30 seconds at 55° C., and

30 seconds at 72° C.,

7 minutes at 72° C.

Then, if the product of the reaction is to be stored, the tube containing the amplicons which were just synthesized is kept at a temperature of 9° C.

III—Analysis of the Amplicons Obtained According to the Invention

1^{o}) Device for Testing an Individual's Genetic Predisposition to Insulin-Dependent Diabetes

A device based on the ELOSA principle has been developed to analyze the HLA-DQB1 marker, in the form of a strip of eight wells similar to those of a microtiter plate, with a detection probe conjugated to peroxidase (POD) for colorimetric visualization.

A—Capture Probes

Specific capture probes are used in each of the eight wells. These probes are actually synthetic oligonucleotides with a 5′ amine bridge as described in the Applicant's Patents U.S. Pat. No. 5,510,084 and EP-B-0.549.776. It is this 5′ amine bridge—with its special structure—which make it possible to immobilize the probes at the base of the well. It is therefore important in the following to understand that this bridge is present at the 5′ end of the probes, even when this is not specified.

The capture probes are immobilized in the wells according to the pattern shown in Table 3; the target alleles corresponding to each capture probe in Table 3 are specified in Table 4 below.

TABLE 3

Distribution of capture probes in wells

| | | bioMérieux |
|---|---|---|
| Sequence n° | Sequence (5′ > 3′) | designation |
| SEQ ID NO 3 | CgCTrCgACAgCgACgTgggg | C+ |
| SEQ ID NO 4 | TATgAAACTTATggggATAC | C− |
| SEQ ID NO 5 | TTCTTgTgAgCAgAAgC | 26c |
| SEQ ID NO 6 | CCgCCTgCCgCCgA | 57f |
| SEQ ID NO 7 | AggggACCCgggCggA | 70b |
| SEQ ID NO 8 | gACgTggAggTgTACC | 45a |
| SEQ ID NO 9 | gCCgCCTgACgCCg | 57e |
| SEQ ID NO 10 | ggggCCCgggCgTC | 70a |
| SEQ ID NO 11 | AggAggACgTgCgC | 37b |
| SEQ ID NO 12 | TCTTgTAACCAgATAC | 26b |
| SEQ ID NO 13 | ggTggACACCgTATgCAg | 70c |

TABLE 4
Possible target alleles for each capture probe

| | | Predisposition |
|---|---|---|
| Sequence n° | HLA-DQB1 specificity* | status |
| SEQ ID NO 3 | All | Not applicable |
| SEQ ID NO 4 | None | Not applicable |
| SEQ ID NO 5 | 0201, 0202, 0203 | Susceptibility |
| SEQ ID NO 6 | 0302, 0304, 0305, 0307, 0308 | Susceptibility |
| SEQ ID NO 7 | 0602, 0603, 0608, 0610, 06111, 06112, | Protection |
| | 0612, 0613, 0614, 0616, 0308 | |
| SEQ ID NO 8 | 03011, 03012, 0304, 0309 | Protection |
| SEQ ID NO 9 | 03011, 03012, 03032, 03033, 0306, | Protection |
| | 0309, 0310 | |
| SEQ ID NO 10 | 05011, 05012, 0502, 05031, 05032 | Neutral |
| SEQ ID NO 11 | 06011, 06012, 06013 | Neutral |
| SEQ ID NO 12 | 06051, 06052, 0606, 0609, 06112, | Neutral |
| | 0612 | |
| SEQ ID NO 13 | 0306, 0401, 0402 | Neutral |

In Table 3, the C+ wells contain a positive control (SEQ ID NO 3) which detects all alleles of the HLA-DQB1* gene; this is to confirm that the relevant locus has been amplified, i.e. the region between the two primers SEQ ID NO 1 and SEQ ID NO 2 described in Table 1. The negative control (SEQ ID NO 4) has no diagnostic value; it is only included to satisfy certain Norms. This sequence is not in any way HLA-specific; it represents a randomly selected sequence which does not occur in any HLA genes.

On the other hand, results with the other probes—SEQ ID NO 5 to SEQ ID NO 13—do have diagnostic value and can be used to estimate an individual's genetic predisposition to disease, more precisely, to insulin-dependent diabetes.

It can be seen that probe SEQ ID NO 5 detects susceptibility to the disease, namely insulin-dependent diabetes. It is specific for major alleles which are known to confer susceptibility to this disease, namely the HLA-DQB1*0201 and HLA-DQB1*0202 alleles. These major alleles are represented in bold in Table 4.

Similarly, probe SEQ ID NO 6 also detects susceptibility to insulin-dependent diabetes. It is specific for another major allele which confers susceptibility to this disease, namely the HLA-DQB1*0302 allele.

Probe SEQ ID NO 7 detects protection against the disease, namely insulin-dependent diabetes. It is specific for major alleles which are known to confer protection against this disease, namely the HLA-DQB1*0602 and HLA-DQB1*0603 alleles.

Similarly, probe SEQ ID NO 8 also detects protection against insulin-dependent diabetes. It is specific for a major allele which confers protection against this disease, namely the HLA-DQB1*0301 allele, be it the HLA-DQB1*03011 or the HLA-DQB1*03012 variant.

Probe SEQ ID NO 9 also detects protection against insulin-dependent diabetes, since it also detects the HLA-DQB1*0301 allele, both the HLA-DQB1*03011 and the HLA-DQB1*03012 variants. The point of this probe is to detect the HLA-DQB1*03032 allele which is fairly common and would be expected to be protective in the light of the amino acid it carries at position 57.

It should be noted that the two probes SEQ ID NO 6 and SEQ ID NO 7—the one corresponding to susceptibility to insulin-dependent diabetes and the other to protection against it—both detect the HLA-DQB1*0308 allele. It is worth noting that this allele is very rare. The chance of finding it all are small, and the chance of finding it in the homozygous state (HLA-DQB1*0308/HLA-DQB1*0308) is almost zero. The same reasoning can be applied with respect to the two probes SEQ ID NO 6 and SEQ ID NO 8—the one corresponding to susceptibility to insulin-dependent diabetes and the other to protection against it. Both these probes detect the HLA-DQB1*0304 allele which is also extremely rare.

In both these cases, two probes, one for susceptibility and the other for protection, can detect the alleles because the prevalence is too low.

B—Detection Probes

The probe used for detection is an oligonucleotide with a 5′ amine bridge conjugated to peroxidase (POD). The sequence of this oligonucleotide is given in Table 5.

TABLE 5

Detection probe for the capture probes used

| | | |
|---|---|---|
| | | bioMérieux |
| Sequence n° | Sequence (5′ > 3′) | designation |

| | | |
|---|---|---|
| SEQ ID NO 14 | TggAACAgCCAgAAggA | D4-POD |

Of course, the sequence of this detection probe was determined in such a way that it is complementary to all the amplicons which can form hybrids with capture probes

C—Procedure

Firstly, preparing the amplicons involves:

transferring all the amplicon-containing solution (50 μl) into a 1.5 ml microfuge tube,

adding 5 μl of reagent R2 (2N NaOH),

mixing well,

incubating for 5 minutes at a temperature of between 18 and 25° C.

adding 1 ml of hybridization buffer, the composition of which is stipulated below,

adding 50 μl of the detection probe solution, the composition of which is stipulated below,

mixing well.

The composition of the hybridization buffer is:

0.1 M sodium phosphate (pH 6.8),

0.5 M NaCl, 2% (m/v) polyethylene glycol 4000,

0.65% (m/v) Tween 20, 0.1% (m/v) gelatin,

0.14 g/L sonicated salmon sperm DNA,

0.2 g/L BND (Bromo-Nitro-Dioxane as preservative), and

0.01 g/L ciprofloxacin.

Detection probe SEQ ID NO 14 (D4-POD) is diluted in 5 mM phosphate buffer (pH 7.0) containing 0.5% (m/v) bovine serum albumin and 0.5% (m/v) phenol.

Secondly, target hybridization is performed as follows:

100 μl aliquots of the mixture are dispensed into each of the eight wells of the R1 strip,

the strip is covered with adhesive film, and

incubate for one hour at 37° C. (37±1° C.).

Thirdly, any unhybridized targets are removed by washing three times with 500 μl aliquots of Color 0 wash buffer diluted twenty-fold with H₂O. “Color 0” means a twenty-fold (20×) concentrate of PBS containing 1% (m/v) Tween 20.

Fourthly, the purpose of the visualization step is determine which capture probes have formed hybrids with a target oligonucleotide. This is achieved in the following way:

into each well, add 100 μl of freshly prepared substrate solution—1 tablet of Color 1 in 5 ml of Color 2,

incubate for 20 minutes in the dark at a temperature of between 18 and 25° C.,

add 50 μl of Color 3, and

read the absorbance of the solution at 492 nm.

“Color 1” means O-phenylenediamine dihydrochloride (OPD); “Color 2” means 0.1 M sodium phosphate, 0.05 M citric acid, 0.03% H₂O₂,; and “Color 3” means 1.8 N H₂SO₄.

Fifthly, the results visualized in the way described above are interpreted in order to define the individual's genetic predisposition to insulin-dependent diabetes. This process involves:

checking that the optical density (OD) of positive control SEQ ID NO 3 (C+) is greater than 0.8, and that the OD of negative control SEQ ID NO 4 (C−) is below 0.05,

and determining the genotype with respect to the two alleles identified by probes SEQ ID NO 5 and SEQ ID NO 13.

In the above-mentioned case, the peroxidase is an enzyme which—in the presence of an appropriate substrate (e.g. OPD)—makes it possible to detect whether or not any of the probes has formed a hybrid with an oligonucleotide. Because this substrate is soluble, the signal will diffuse. In this case, in order to make it possible to detect whether the subject is homozygous or heterozygous, and whether his/her alleles confer susceptibility to or protection against insulin-dependent diabetes, or whether they are neutral in that respect, it is necessary to include a capture probe in the compartment, or in a simpler way, in the well. This is true for the probes (SEQ ID NO 5 and 6) related to susceptibility to insulin-dependent diabetes, and for those (SEQ ID NO 7, 8 and 9) related to protection against this disease. On the other hand, the probes (SEQ ID NO 10, 11, 12 and 13) which are neutral vis-à-vis predisposition to this disease can be grouped together without any problem.

This is not true if the substrate used precipitates out of solution or if the detection probe (SEQ ID NO 14) is fluorescently labeled. In these cases, several different probes can be included in the same compartment or well, even if the combination contains both susceptibility-related (SEQ ID NO 5 or 6) and protection-related (SEQ ID NO 7, 8 or 9) probes. In practice, in its most compact configuration, all the probes related to susceptibility, protection and neutrality can be included in the same compartment or well, with the only limiting factor being the facility with which the operator can group different types of probe without any overlap between areas carrying different capture oligonucleotides. As stated above, a device in which this type of solid substrate can be implemented has already been described in a Patent Application submitted by the Applicant on Nov. 15, 2000 under Number FR00/14691.

2^{o}) Examples of Tests Carried Out According to the Invention

A—Sample 1

Table 6 below shows the results obtained using a R1 strip with each well containing an aliquot of a sample derived from the first test patient.

TABLE 6
Results obtained with an R1 strip for the first sample

| | OD (X | |
|---|---|---|
| R1 strip | 1,000) | +/− |
| SEQ ID NO 3 (C+) | >2500 | + |
| SEQ ID NO 4 (C−) | 0 | − |
| SEQ ID NO 5 (S) | 1245 | + |
| SEQ ID NO 6 (S) | 126 | + |
| SEQ ID NO 7 (P) | 7 | − |
| SEQ ID NO 8 (P) | 9 | − |
| SEQ ID NO 9 (P) | 1 | − |
| SEQ ID NO 10 through | 8 | − |
| 13 (N) | | |

The analysis is as follows:

the SEQ ID NO 3 probe gives a positive result: the HLA-DQB1 gene has been amplified and hybridization has occurred as it should,

the SEQ ID NO 5 probe gives a positive result: there is a HLA-DQB1*0201 or HLA-DQB1*0202 or HLA-DQB1*0203 allele present,

the SEQ ID NO 6 probe gives a positive result: there is a HLA-DQB1*0302 or HLA-DQB1*0304 or HLA-DQB1*0305 or HLA-DQB1*0307 or HLA-DQB1*0308 allele present, and

the other probes give negative results.

In conclusion, there is a strong probability that the subject is carrying two alleles which confer susceptibility to insulin-dependent diabetes. Luckily, it is important to realize that the susceptibility-conferring HLA-DQB1*0302 allele is far more common than the neutral alleles HLA-DQB1*0304, HLA-DQB1*0305, HLA-DQB1*0307 and HLA-DQB1*0308.

These two HLA-DQB1 alleles were detected in a generic test; the actual HLA genotype of this subject was:

HLA-DQB1*02, and

HLA-DQB1*0302.

B—Sample 2

Table 7 below shows the results obtained using a R1 strip with each well containing an aliquot of a sample derived from the second test patient.

TABLE 7
Results obtained with an R1 strip for the second sample

| | OD (X | |
|---|---|---|
| R1 strip | 1,000) | +/− |
| SEQ ID NO 3 (C+) | 1685 | + |
| SEQ ID NO 4 (C−) | 5 | − |
| SEQ ID NO 5 (S) | 708 | + |
| SEQ ID NO 6 (S) | 22 | − |
| SEQ ID NO 7 (P) | 7 | − |
| SEQ ID NO 8 (P) | 143 | + |
| SEQ ID NO 9 (P) | 41 | − |
| SEQ ID NO 10 through | 11 | − |
| 13 (N) | | |

The analysis is as follows:

the SEQ ID NO 3 probe gives a positive result: the HLA-DQB1 gene has been amplified and hybridization has occurred as it should,

the SEQ ID NO 5 probe gives a positive result: there is a HLA-DQB1*0201 or HLA-DQB1*0202 or HLA-DQB1*0203 allele present,

the SEQ ID NO 8 probe gives a positive result: there is a HLA-DQB1*03011 or HLA-DQB1*03012 or HLA-DQB1*0304 or HLA-DQB1*0309 allele present, and

the other probes give negative results.

In conclusion, there is a strong probability that the subject is carrying one allele which confers susceptibility to insulin-dependent diabetes, with his/her other allele conferring protection against this disease. Luckily, it is important to realise that the susceptibility-conferring HLA-DQB1*03011 et HLA-DQB1*03012 alleles are far more common than the neutral alleles HLA-DQB1*0304 and HLA-DQB1*0309.

These two HLA-DQB1 alleles were detected in a generic test; the actual HLA genotype of this subject was:

HLA-DQB1*02, and

HLA-DQB1*0301.

C—Sample 3

Table 8 below shows the results obtained using a R1 strip with each well containing an aliquot of a sample derived from the third test patient.

TABLE 8
Results obtained with an R1 strip for the third sample

| | OD (X | |
|---|---|---|
| R1 strip | 1,000) | +/− |
| SEQ ID NO 3 (C+) | >2500 | + |
| SEQ ID NO 4 (C−) | 0 | − |
| SEQ ID NO 5 (S) | 1790 | + |
| SEQ ID NO 6 (S) | 27 | − |
| SEQ ID NO 7 (P) | 31 | − |
| SEQ ID NO 8 (P) | 19 | − |
| SEQ ID NO 9 (P) | 15 | − |
| SEQ ID NO 10 through | 829 | + |
| 13 (N) | | |

The analysis is as follows:

the SEQ ID NO 3 probe gives a positive result: the HLA-DQB1 gene has been amplified and hybridization has occurred as it should,

the SEQ ID NO 5 probe gives a positive result: there is a HLA-DQB1*0201 or HLA-DQB1*0202 or HLA-DQB1*0203 allele present,

the SEQ ID NO 10 through 13 probes give a positive result: there is a HLA-DQB1*0306 or HLA-DQB1*0401 or HLA-DQB1*0402 or HLA-DQB1*05011 or HLA-DQB1*05012 or HLA-DQB1*0502 or HLA-DQB1*05031 or HLA-DQB1*05032 or HLA-DQB1*06011 or HLA-DQB1*06012 or HLA-DQB1*06013 or HLA-DQB1*06051 or HLA-DQB1*06052 or HLA-DQB1*0606 or HLA-DQB1*0609 or HLA-DQB1*06112 or HLA-DQB1*0612 allele present, and

the other probes give negative results.

In conclusion, the subject is definitely carrying one allele which confers susceptibility to insulin-dependent diabetes, with his/her other allele being neutral vis-à-vis predisposition to this disease. Once it has been ascertained that the subject is carrying a neutral allele, there is little point in trying to determine exactly which allele is concerned in the context of the test according to this invention.

These two HLA-DQB1 alleles were detected in a generic test; the actual HLA genotype of this subject was:

HLA-DQB1*02, and

HLA-DQB1*0501.

IV—Other Approaches to Improve the Invention (Non-Limiting)

1^{o}) Using PCR Amplicons Made with Biotinylated Primers

A—Biotinylated Primers

The same set of specific capture probes used to determine a subject's genetic predisposition to insulin-dependent diabetes (Chapter III, 1^{o}) can also be used with biotinylated amplicons, made by using PCR primers carrying a biotin group at their 5′ end (see Table 9 below).

TABLE 9

Biotinylated primers for amplification of the HLA-DQB1 gene

| Sequence n° | Sequence (5′ > 3′) | Scientific name |
|---|---|---|
| Biotin-SEQ ID NO 1 | Biotin-CATGTGCTACTTCACCAACGG | DQBAMP-A-Biotin |
| Biotin-SEQ ID NO 2 | Biotin-CTGGTAGTTGTGTCTGCACAC | DQBAMP-B-Biotin |

Although the conditions for amplification are identical to those described above, the procedure differs.

B—Procedure with Biotinylated Amplicons

Firstly, preparing the amplicons involves:

transferring all the amplicon-containing solution (50 μl) into a 1.5 ml microfuge tube,

adding 5 μl of reagent R2 (2N NaOH),

mixing well,

incubating for 5 minutes at a temperature of between 18 and 25° C.

adding 1 ml of hybridization buffer, the composition of which is stipulated below, and

mixing well.

The composition of the hybridization buffer is:

0.1 M sodium phosphate (pH 6.8),

0.5 M NaCl, 2% (m/v) polyethylene glycol 4000,

0.65% (m/v) Tween 20, 0.1% (m/v) gelatin,

0.14g/L sonicated salmon sperm DNA,

0.2 g/L BND (biocide or Bromo-Nitro-Dioxane), and

0.01 g/L ciprofloxacin.

Detection probe SEQ ID NO 14 (D4-POD) is diluted in 5 mM phosphate buffer (pH 7.0) containing 0.5% (m/v) bovine serum albumin and 0.5% (m/v) phenol.

Secondly, target hybridization is performed as follows:

100 μl aliquots of the mixture are dispensed into each of the eight wells of the R1 strip,

the strip is covered with adhesive film, and

incubated for one hour at 37° C. (37±1° C.).

Thirdly, any unhybridized targets are removed by washing three times with 500 μl aliquots of Color 0 wash buffer diluted twenty-fold with H₂O.

Fourthly, the purpose of the visualization step is determine which capture probes have formed hybrids with a target oligonucleotide. This is achieved in the following way:

add 100 μl of a solution of a streptavidin-peroxidase conjugate (SPOD, Rache, ref. 1089153) diluted 10,000-fold in PBS buffer containing 0.1% Tween 20 and 0.02% bovine serum albumin (pH 7.2),

incubate for 30 minutes in the dark at a temperature of between 18 and 25° C.,

wash three times with 500 μl aliquots of Color 0 wash buffer diluted twenty-fold with H₂O

into each well, add 100 μl of freshly prepared substrate solution—1 tablet of Color 1 in 5 ml of Color 2,

incubate for 20 minutes in the dark at a temperature of between 18 and 25° C.,

add 50 μl of Color 3, and

read the absorbance of the solution at 492 nm.

“Color 0”, “Color 1”, “Color 2” and “Color 3” are exactly the same as described in the preceding section.

Fifthly, the results visualized in the way described above are interpreted in order to define the individual's genetic predisposition to insulin-dependent diabetes. This process involves:

checking that the optical density (OD) of positive control SEQ ID NO 3 (C+) is greater than 0.8, and that the OD of negative control SEQ ID NO 4 (C−) is below 0.05,

and determining the genotype with respect to the two alleles identified by probes SEQ ID NO 5 and SEQ ID NO 13.

This protocol takes longer and is slightly more complex since it includes an extra step, but it gives more intense signals for the weaker probes, notably SEQ ID NO 6.

Table 10 below effectively illustrates the differences between results obtained with biotinylated amplicons and those obtained with uinbiotinylated ones.

| Probe | Un-biotinylated amplicons | Biotinylated amplicons |
|---|---|---|
| SEQ ID NO 3 (C+) | >2500 | >2500 |
| SEQ ID NO 4 (C−) | 0 | 0 |
| SEQ ID NO 5 (S) | 1 | 14 |
| SEQ ID NO 6 (S) | 134 | 324 |
| SEQ ID NO 7 (P) | 15 | 54 |
| SEQ ID NO 8 (P) | 11 | 34 |
| SEQ ID NO 9 (P) | 19 | 35 |
| SEQ ID NO 10 | 603 | 1322 |
| through 13 (N) | | |

FIG. 10 OD readings (×1,000) made with a HLA-DQB1*0302/0605 heterozygous cell line

It should be noted that, with biotinylated amplicons, use is no longer made of the 5′ amine bridge included to make it possible to immobilize the detection probe at the bottom of the well, as described above; this is because the streptavidin conjugated to the peroxidase can bind the biotin present on the amplicons thereby capturing them.

2^{o}) Using Capture Probes Immobilized on Beads

In order to enhance the specific signal in wells in which, for example, the SEQ ID NO 6 probe is present, it is possible to use a coating furnished with 100 μl of polystyrene beads with a diameter of 0.1 mm. These beads can be bought from Polysciences (Warrington, Pa., USA, ref.: 00876). They are coated in avidin from Sigma (Saint-Louis, Mo., USA, ref.: A9275) so they bind capture probe SEQ ID NO 6 which has been biotinylated at its 5′ end. In this case, due to the presence of the biotin, the specific capture oligonucleotide SEQ ID NO6 does not carry a bridge.

A—Bead Preparation

1—Preparation of Avidin-Coated Beads

The beads are prepared by:

diluting 2.5 μl of a solution of 5 mg/ml avidin in 100 μl of 50 mM borate buffer (pH 9.3),

adding 7.4 μl of the bead suspension, and

incubating for 30 minutes at a temperature of 37C.

2—Preparation of Avidin-Coated Beads Carrying Capture Probes

In addition:

add 5.10¹⁵ molecules of the biotinylated SEQ ID NO 6 probe, and

incubate for 30 minutes at a temperature of 37C.

B—Coating with Avidin-Coated Beads Carrying Capture Probes

Aliquots of 100 μl of a ten-fold dilution of coated beads in coating buffer (3× PBS) are then added to the wells. The coating conditions used for amine bridge-containing probes are the same as those used for their adsorption to the bottom of the well, i.e. 2 hours at a temperature of 37° C., or 125 hours at a temperature of between 18 and 25° C.

This measure also helps enhance the signal in certain wells, e.g. those containing the SEQ ID NO 6 probe, in order to facilitate analysis of those wells. The following OD readings were obtained with the SEQ ID NO 6 probe with a cell line homozygous for HLA-DQB1*0302:

no beads: 172, and

with beads: 689.

3^{o}) Using a Single Well for Capture Probes Specific for Alleles Which are Neutral vis-à-vis Predisposition to Insulin-Dependent Diabetes

However, if peroxidase is used to visualize hybrids formed between target oligonucleotides and capture oligonucleotides, a single well can be used to analyze the “neutrality of alleles” with respect to insulin-dependent diabetes. This involves a set of four specific capture probes (SEQ ID NO 10 through 13), each of which has its own specificity.

As mentioned above, if a technique to deposit multiple spots at the bottom of a single well of a microtiter plate (e.g. a 96-well microtiter plate) is developed, the four probes mentioned above could be spotted separately in a single well.

4^{o}) Using a Single Well for the Capture Probes Specific for All Alleles Involved in Insulin-Dependent Diabetes

If a technique to deposit multiple spots at the bottom of a single well of a microtiter plate can be perfected, all eleven (11) capture probes—both non-specific (SEQ ID NO 3 and 4) and specific (SEQ ID NO 5 to 13) as described above in Tables 3 and 4—could be spotted in the same well. Performing hybridization of the amplicons in a single well brings a number of advantages:

the test is more reliable since all eleven (11) probes come into contact with all the amplicons at the same time, thereby eliminating the risk of false negatives,

the test is more sensitive since all eleven (11) probes come into contact with all the amplicons in the sample, and

more tests can be carried out in the same number of wells, i.e. a total of ninety-six (96) tests can be carried out on a single microtiter plate; this is of particular worth for screening applications.

As mentioned above, this approach necessarily entails using a topologically restricted visualization system to detect specific hybrids formed at the bottom of the well. By way of example, the property of fluorescence could be used to detect amplicons into which either some fluorescent species or a molecule which can be visualized using a fluorescent label has been incorporated.

Feasibility tests to investigate the possibility of depositing several spots in a single well have been carried out, using a calorimetric visualization system.

A—Spotting

Tests were carried out in which four (4) spots corresponding to specific capture probes SEQ ID NO 10 through 13 (the form containing an amine bridge) were deposited by hand. The spots consisted of 1 and 5 μl of a solution containing 400-1000 pmoles/ml in coating buffer (3× PBS). The spots were incubated for between 15 minutes and two hours at a temperature of 37° C., or for 15 hours at a temperature of between 18 and 25° C.

B—Washing

The wells were washed using diluted Color 0.

C—Target Preparation

PCR amplicons were prepared as described in the preceding section (III—1^{o})—C.

The following steps were carried out in the amplification tube:

a 50 μl aliquot of the amplicons was denatured by adding 1 μl of 2N NaOH,

this was incubated for 5 minutes at a temperature of between 18 and 25° C.,

40 μl of hybridization buffer (15× SSPE, 2% PEG 4000, 1.5% Tween 20, 0.22% gelatin, 0.032% sonicated DNA and preservatives) were added,

and then a 6 μl aliquot of a solution of the D4-POD conjugate (50 pmoles/ml in phosphate buffer with 0.5% [m/v] BSA and 0.5% [m/v] phenol [ph 7.0]) was added.

D—Hybridization:

All the reaction mixture was transferred into a well containing spots of the four (4) capture probes, and this was incubated for one hour at a temperature of 37±1° C.

B—Washing

Washing was carried out using diluted Color 0.

F—Visualization

The following steps were carried out in order:

a 100 μl aliquot of substrate was added (Color 1 diluted in Color2),

the reaction was allowed to proceed for 20 minutes in the dark without any perturbation at a temperature of between 18 and 25° C.,

the color at the location of the positive probe was observed,

to quantify the signal, an aliquot of 50 μl of Color 3 was added,

the solution was mixed by gently shaking, and

its OD was read at 492 nm.

### Literature References Included in this Description as a Complement

Benjamin, 1990:

Benjamin R, Parham P, Immunology Today, 1990, 11, 137-142, Guilt by association: HLA-B27 and ankylosing spondylitis

Brewerton, 1973:

Brewerton D A, Caffrey M, Hart F D et al, Lancet, 1973, 1, 904-907. Ankylosing spondylitis and HL-A27.

Gregersen, 1986:

Gregersen P K, Shen M, Song Q L, Merryman P, Degar S, Seki T, Maccari J, Goldberg D, Murphy H, Schwenzer J, Wang C Y, Winchester R J, Nepom G T, Silver J, Proc. Natl. Acad. Sci. USA, 1986, 83, 2642-2646. Molecular diversity of HLA-DR4 haplotypes.

Gregersen, 1987:

Gregersen P K, Silver J, Winchester R J, Arthritis Rheum., 1987, 30, 1205-1213. The shared epitope hypothesis. An approach to understanding the molecular genetics of susceptibility to rheumatoid arthritis.

Hiraiwa, 1990:

Hiraiwa A, Yamanaka K, Kwok W W, Mickelson E M, Masewicz S, Hansen J A, Radka S F, Nepom G T, Proc. Natl. Acad. Sci. USA, 1990, 87, 8051-8055. Structural requirements for recognition of the HLA-Dw14 class II epitope: a key HLA determinant associated with rheumatoid arthritis.

Khan, 1979:

Khan M A, Kushner I, Ballou S P et al, Lancet, 1979, 1, 921-922, Familial rheumatoid arthritis and HLA-DRW4.

Komulainen, 1999

Komulainen J, Kulmala P, Savola K, Lounamaa R, Ilonen J, Reijonen H, Knip M, Akerblom H K; Diabetes Care 1999, vol 22, n^{o}12, 1950-1955

Kulmala, 2000

Kulmala P, Savola K, Reijonen H, Veijola R, Vahasalo P, Karjalainen J, Thuomiletho-Wolf E, Ilonen J, Tuomiletho J, Akerblom H K, Knip M; Diabetes 2000, vol 49, 48-58

Lawrence, 1970:

Lawrence J, Ann. Rheum. Dis., 1970, 29, 357-379

Nepom, 1989:

Nepom G T, Byers P, Seyfried C, Healey L A, Wilske K R, Stage D, Nepom B S, Arthritis Rheum. 1989, 32, 15-21. HLA genes associated with rheumatoid arthritis. Identification of susceptibility alleles using specific oligonucleotide probes.

Nepom, 1991:

Nepom G T, Erlich H, Annu. Rev. Immunol., 1991, 9, 493-525. MHC class-II molecules and autoimmunity.

Schlosstein, 1973:

Schlosstein L, Terasaki P I, Bluestone R, Pearson C M, N. Engl. J. Med., 1973, 288, 704-706. High association of a HL-A antigen, w27, with ankylosing spondylitis

Stastny, 1978:

Stastny P, N. Engl. J. Med., 1978, 298, 869-871. Association of the B-cell alloantigen DRw4 with rheumatoid arthritis.

Stastny, 1983:

Stastny P, Ball E J, Dry P J, Nunez G, Immunol. Rev., 1983, 70, 113-154. The human immune response region (HLA-D) and disease susceptibility.

Stastny, 1988:

Stastny P, Ball E, Kahn M, Olsen N, Pincus T, Gao X, Br. J. Rheumatol., 1988, 27, 132-138. HLA-DR4 and other genetic markers in rheumatoid arthritis.

Todd, 1988:

Todd J A, Acha-Orbea H, Bell J I, Chao N, Fronek Z, Jacob C O, McDermott M, Sinha A A, Timmerman L, McDevitt H O, Science, 1988, 240, 1003-1009

Wordsworth, 1989:

Wordsworth B P, Lanchbury J S, Sakkas L I, Welsh K I, Panayi G S, Bell J I, Proc. Natl. Acad. Sci. USA, 1989, 86, 10049-10053

1. A method for testing a subject's genetic predisposition to an autoimmune disease, consisting in taking a liquid sample containing at least one type of amplicon generated by the amplification of at least one polymorphic region relevant to the disease concerned, and adding to it probes selected in the following way:
at least one probe which is specific for the subject's susceptibility to the disease,
at least one probe which is specific for said subject's protection against said disease,
at least one probe which is specific for said subject's neutral status vis-à-vis predisposition to said disease,
and consisting in visualizing any hybrids formed.

2. The method according to claim 1, in which the autoimmune disease is a form of insulin-dependent diabetes.

3. The method according to either of claims 1 or 2, characterized in that the probes used to detect a subject's predisposition to insulin-dependent diabetes are defined as follows:
at least one probe which is specific for the susceptibility alleles HLA-DQB1*0201, HLA-DQB1*0202 and HLA-DQB1*0302,
at least one probe which is specific for the protective alleles HLA-DQB1*0301, HLA-DQB1*0602 and HLA-DQB1*0603,
at least one probe which is specific for other alleles which are neutral vis-à-vis a subject's predisposition to insulin-dependent diabetes.

4. The method according to any of claims 1 through 3, characterized in that the probes used to detect a subject's predisposition to insulin-dependent diabetes are defined as follows:
at least one probe which is specific for the susceptibility alleles HLA-DQB1*0201, HLA-DQB1*0202, HLA-DQB1*0203, HLA-DQB1*0302, HLA-DQB1*0304, HLA-DQB1*0305, HLA-DQB1*0307 and HLA-DQB1*0308,
at least one probe which is specific for the protective alleles HLA-DQB1*03011, HLA-DQB1*03012, HLA-DQB1*03032, HLA-DQB1*03033, HLA-DQB1*0304, HLA-DQB1*0306, HLA-DQB1*0308, HLA-DQB1*0309, HLA-DQB1*0310, HLA-DQB1*0602, HLA-DQB1*0603, HLA-DQB1*0608, HLA-DQB1*0610, HLA-DQB1*06111, HLA-DQB1*06112, HLA-DQB1*0612, HLA-DQB1*0613, HLA-DQB1*0614 and HLA-DQB1*0616, and
at least one probe which is specific for the neutral alleles HLA-DQB1*0306, HLA-DQB1*0401, HLA-DQB1*0402, HLA-DQB1*05011, HLA-DQB1*05012, HLA-DQB1*0502, HLA-DQB1*05031, HLA-DQB1*05032, HLA-DQB1*06011, HLA-DQB1*06012, HLA-DQB1*06013, HLA-DQB1*06051, HLA-DQB1*06052, HLA-DQB1*0606, HLA-DQB1*0609, HLA-DQB1*06112 and HLA-DQB1*0612.

5. The method according to any of claims 1 through 4, characterized in that the probes used to detect a subject's predisposition to insulin-dependent diabetes are defined as follows:
a probe which is specific for the alleles HLA-DQB1*0201, HLA-DQB1*0202 and HLA-DQB1*0203, and
a probe which is specific for the alleles HLA-DQB1*0302, HLA-DQB1*0304, HLA-DQB1*0305, HLA-DQB1*0307 and HLA-DQB1*0308.

6. The method according to claim 5, characterized in that the probes used to detect susceptibility to insulin-dependent diabetes consist of at least ten (10) nucleotides linked to form the following sequences:

| | |
|---|---|
| (TCTTgTgAgCAgAAgC), et | SEQ ID NO 5 |
| (CCgCCTgCCgCCgA). | SEQ ID NO 6 |

7. The method according to any of claims 1 through 6, characterized in that the probes used to detect protection against insulin-dependent diabetes are defined as follows:
a probe which is specific for the alleles HLA-DQB1*03011, HLA-DQB1*03012, HLA-DQB1*0304 and HLA-DQB1*0309,
a probe which is specific for the alleles HLA-DQB1*03011, HLA-DQB1*03012, HLA-DQB1*03032, HLA-DQB1*03033, HLA-DQB1*0306, HLA-DQB1*0309 and DQB1*0310, and
a probe which is specific for the alleles HLA-DQB1*0308, HLA-DQB1*0602, HLA-DQB1*0603, HLA-DQB1*0608, HLA-DQB1*0610, HLA-DQB1*06111, HLA-DQB1*06112, HLA-DQB1*0612, HLA-DQB1*0613, HLA-DQB1*0614 and HLA-DQB1*0616.

8. The method according to claim 7, characterized in that the probes used to detect protection against insulin-dependent diabetes consist of at least ten (10) nucleotides linked to form the following sequences:

| | |
|---|---|
| (AggggACCCgggCggA), | SEQ ID NO 7 |
| (gACgTggAggTgTACC), et | SEQ ID NO 8 |
| (gCCgCCTgACgCCg). | SEQ ID NO 9 |

9. The method according to any of claims 1 through 8, characterized in that the probes used to detect neutral status vis-à-vis predisposition to insulin-dependent diabetes are defined as follows:
a probe which is specific for the alleles HLA-DQB1*0306, HLA-DQB1*0401 and HLA-DQB1*0402, and
a probe which is specific for the alleles HLA-DQB1*05011, HLA-DQB1*05012, HLA-DQB1*0502, HLA-DQB1*05031 and HLA-DQB1*05032,
a probe which is specific for the alleles HLA-DQB1*06011, HLA-DQB1*06012 and HLA-DQB1*06013, and
a probe which is specific for the alleles HLA-DQB1*06051, HLA-DQB1*06052, HLA-DQB1*0606, HLA-DQB1*0609, HLA-DQB1*06112 and HLA-DQB1*0612.

10. The method according to claim 9, characterized in that the probes used to detect neutral status vis-à-vis predisposition to insulin-dependent diabetes consist of at least ten (10) nucleotides linked to form the following sequences:

| | |
|---|---|
| (ggggCCCgggCgTC), | SEQ ID NO 10 |
| (AggAggACgTgCgC), | SEQ ID NO 11 |
| (TCTTgTAACCAgATAC), and | SEQ ID NO 12 |
| (ggTggACACCgTATgCAg). | SEQ ID NO 13 |

11. The method according to any of claims 4 through 10, characterized in that at least one positive control probe capable of hybridizing with all HLA-DQB1 genes is used to detect all HLA-DQB1 alleles.

12. The method according to any of claims 6, 8, 10 or 11, characterized in that a maximum of 38.89%, preferably no more than 20%, of the bases in any of the probes are substituted by at least one similar base such as inosine.

13. The method according to any of claims 1 through 12, characterized in that the polymorphic region or regions relevant to the disease are amplified in a preliminary step.

14. The method according to any of claim 13, characterized in that the primers for amplification are biotinylated in such a way that the resultant amplicons are likewise biotinylated.

15. The method according to either of claims 13 or 14, characterized in that, prior to amplification, the biological specimen—preferably in the form of a dry spot of blood—is processed in order to extract the nucleic acid therein.

16. The method according to claim 15, characterized in that the nucleic acid is extracted into a reaction mixture which already contains the deoxynucleotide triphosphates (dNTPs) to be used in the amplification reaction, and this prior to incubation.

17. The method according to claim 16, characterized in that, after incubation, deoxynucleotide triphosphates (dNTPs)—also to be used for the subsequent amplification step—are added into the reaction mixture.

18. A device for implementing the method according to any of claims 1 through 17, characterized in that each type of probe is immobilized in a separate compartment (e.g. the well of a microtiter plate), apart from the other probes.

19. The device according to claim 18, characterized in that each type of probe used to detect susceptibility to or protection against insulin-dependent diabetes is immobilized in a separate compartment (e.g. the well of a microtiter plate), apart from the other probes used to detect susceptibility or protection, and that all or a fraction of the various types of probe used to detect neutrality vis-à-vis a subject's predisposition to insulin-dependent diabetes are immobilized in at least one well of a microtiter plate.

20. The device according to either of claims 18 or 19, characterized in that at least two types of different, specific probe are immobilized in a single compartment (e.g. the same well of a microtiter plate) without any interaction occurring between them.

21. The device according to either of claims 18 or 19, characterized in that all the different types of probe used to detect susceptibility to, protection against, and neutrality vis-à-vis predisposition to insulin-dependent diabetes are immobilized in a single compartment (e.g. the same well of a microtiter plate).

22. A method for visualizing hybrids formed in a device according to either of claims 18 or 20, characterized in that visualization depends on an unlocalized calorimetric reaction catalyzed by an enzyme, e.g. peroxidase.

23. The method for visualizing hybrids formed in a device according to any of claims 19 through 21, characterized in that visualization depends on topologically restricted signals so that the hybrids formed between each different type of probe and its corresponding amplicons can be resolved, e.g. by means of signals based on fluorescent or radioactive labels.

24. Primers for the amplification of a sequence corresponding to the HLADQB1 gene, designed for use in a method to test for a subject's genetic predisposition to insulin-dependent diabetes, which involves using a SEQ ID NO 1 primer in conjunction with a SEQ ID NO 2 primer.

25. The primers according to claim 24, characterized in that they are biotinylated at their 5′ end.

26. Capture probes which can hybridize with sequences corresponding to the HLA-DQB1 gene, designed for the testing of a subject's genetic predisposition to insulin-dependent diabetes, which are immobilized either at the bottom of the well of a microtiter plate or on a bead through an amine or biotin bridge located at the 5′ end of said probes.


SEQUENCE LISTING (14 sequences)

SEQ ID NO: 1 - DNA, 21 bp, Homo sapiens
catgtgctacttcaccaacgg

SEQ ID NO: 2 - DNA, 21 bp, Homo sapiens
ctggtagttgtgtctgcacac

SEQ ID NO: 3 - DNA, 21 bp, Homo sapiens
cgcttcgacagcgacgtgggg

SEQ ID NO: 4 - DNA, 20 bp, Homo sapiens
tatgaaacttatggggatac

SEQ ID NO: 5 - DNA, 16 bp, Homo sapiens
tcttgtgagcagaagc

SEQ ID NO: 6 - DNA, 14 bp, Homo sapiens
ccgcctgccgccga

SEQ ID NO: 7 - DNA, 16 bp, Homo sapiens
aggggacccgggcgga

SEQ ID NO: 8 - DNA, 16 bp, Homo sapiens
gacgtggaggtgtacc

SEQ ID NO: 9 - DNA, 14 bp, Homo sapiens
gccgcctgacgccg

SEQ ID NO: 10 - DNA, 14 bp, Homo sapiens
ggggcccgggcgtc

SEQ ID NO: 11 - DNA, 14 bp, Homo sapiens
aggaggacgtgcgc

SEQ ID NO: 12 - DNA, 16 bp, Homo sapiens
tcttgtaaccagatac

SEQ ID NO: 13 - DNA, 18 bp, Homo sapiens
ggtggacaccgtatgcag

SEQ ID NO: 14 - DNA, 17 bp, Homo sapiens
tggaacagccagaagga